(19) United States
(12) Patent Application Publication
(10) Pub. No.: US 2025/0303164 A1
(43) Pub. Date: Oct. 2, 2025

(54) SYSTEM AND METHOD FOR IMPLANTABLE CLOSED-LOOP, BI-DIRECTIONAL BRAINSTEM/SPINAL CORD-MACHINE INTERFACE

(71) Applicant: Ecate LLC, Los Angeles, CA (US)

(72) Inventor: Alessandro MAGGI, Los Angeles, CA (US)

(21) Appl. No.: 19/006,596

(22) Filed: Dec. 31, 2024

Related U.S. Application Data
(60) Provisional application No. 63/570,206, filed on Mar. 26, 2024.

(51) Int. Cl. A61N 1/36

(52) U.S. Cl. CPC A61N 1/36062; A61N 1/36057; A61N 1/36139

(57) ABSTRACT

A spinal cord machine interface (SCMI) device is described. The SCMI includes an intraspinal probe, composed of at least one implantable shank. The implantable shank include a sensing electrode array and a stimulating electrode array. The SCMI also includes an application specific integrated circuit (ASIC). The ASIC is configured to detect action potentials from a patient's spine using the sensing electrode array and to stimulate a target action of the patent using the stimulating electrode array according to a location of the action potentials in a spinal cord and a learned mapping of the location of the action potentials to the target action.

### CROSS-REFERENCE TO RELATED APPLICATION

The present application claims the benefit of U.S. Provisional Patent Application No. 63/570,206, filed Mar. 26, 2024, and titled “SYSTEM AND METHOD FOR IMPLANTABLE CLOSED-LOOP, BI-DIRECTIONAL BRAINSTEM/SPINAL CORD-MACHINE INTERFACE,” the disclosure of which is expressly incorporated by reference herein in its entirety.

### BACKGROUND

### Field

Certain aspects of the present disclosure generally relate to systems and methods for an implantable closed-loop, bi-directional brainstem/spinal-cord machine interface.

### Background

Establishing reliable correlations between one's physiological signals and the associated cognitive and/or psychological states may enable valuable and desired applications for various uses. For example, medical applications as well as consumer electronics domains, amongst others. Such correlations, extensively explored in fundamental sciences, are the focus of various translational attempts into specialized applications such as assessment of cognitive impairment as well as enabling the physically impaired to communicate.

Several factors may be used to determine sensory and/or cognitive information about a subject. For example, such factors may include the type of physiological signals and/or behavioral responses to detect and measure, the type of stimuli to evoke the subject's response, duration of the stimuli, inter-stimuli interval, number of repetitions of each presentation of stimuli, the levels of the stimuli (e.g., sound, brightness or contrast levels, etc.), markers associated with the onset of presentation of each stimuli, etc., as well as the recording sensors and systems. Additionally, the physiological parameters of use (e.g., voltage, power, frequency, etc.), the related time window for analysis, and the analysis structure that can affect the brain signal recordings and correlated cognitive assessment are significant factors. Deviations or mistakes from one or multiples of these parameters can make the difference between a useful or an artifact driven, useless device, system, application, and/or method.

Current brain-machine interfaces (BMI) that aim to provide effective therapies for patients with paralysis mostly target the brain. Despite impressive results, such devices hit critical roadblocks that are inherent to the brain's architecture. In particular, the brain's architecture involves neurons in the cortex that form an extremely complex three-dimensional network that is partially mapped. For example, any given cortical neuron synapse is often competing with thousands of other excitatory and inhibitory neuron synapses. Due to this competitive complexity, recording from any given neuron fails to provide a sufficient, absolute value signal. As a result, a relatively large body of neurons is recorded to enable deciphering of neuronal patterns that are ultimately associated with a target behavior. Consequently, forming a behavioral-neural interface match involves significant patient participation with a medical team, which is time consuming, computationally expensive, power hungry, and heavily reliant on patient compliance.

There is a current and urgent need for a neural device that can address many of these drawbacks.

### SUMMARY

A spinal cord machine interface (SCMI) device is described. The SCMI includes an intraspinal probe, composed of at least one implantable shank. The implantable shank include a sensing electrode array and a stimulating electrode array. The SCMI also includes an application specific integrated circuit (ASIC). The ASIC is designed to detect action potentials originating from the efferent portion of the patient's spinal cord using the sensing array and to stimulate the afferent portion of the patient's spinal cord using the stimulating electrode array, thereby restoring the sensation of pain, temperature, and pressure. A functional map linking specific regions of the spinal cord to their respective sensory and motor functions is generated through the combined sensing and stimulation capabilities of the array. This map enables the system to interpret the patient's motor intentions and selectively stimulate sensory responses corresponding to different areas of the body.

A method for a central nervous system (CNS) transfer is described. The method includes implanting a bi-directional intraspinal cord probe in a target area at the cervical or lower brainstem level of a patient's CNS. The method also includes performing sensory and motor mapping to establish a map between an electrode site of the bi-directional intraspinal cord probe and a corresponding physiological function of the patient. The method further includes transplanting the central nervous system (CNS) of the patient to a synthetic body. The method also includes forming a spinal cord machine interface (SCMI) to the CNS in the synthetic body.

A method for bladder control is described. The method includes monitoring, using a bladder sensing unit, the bladder status until the bladder status indicates a bladder volume within a predetermined percentage of a predetermined maximum bladder volume. The method also includes stimulating, using a stimulator electrode array of an intraspinal probe, a patient for a predetermined amount of time to indicate the bladder status regarding the bladder volume within the predetermined percentage of the predetermined maximum bladder volume. The method further includes detecting, using a sensing electrode array of the intraspinal probe, action potentials in the efferent/motor micturition center of the patient. The method also includes activating a sacral nerve anterior root stimulator (SARS) to void a bladder of the patient in response to the detecting.

This has outlined, rather broadly, the features and technical advantages of the present disclosure in order that the detailed description that follows may be better understood. Additional features and advantages of the present disclosure is described below. It should be appreciated by those skilled in the art that this present disclosure may be readily utilized as a basis for modifying or designing other structures for carrying out the same purposes of the present disclosure. It should also be realized by those skilled in the art that such equivalent constructions do not depart from the teachings of the present disclosure as set forth in the appended claims. The novel features, which are believed to be characteristic of the present disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following description taken in conjunction with the accompanying drawings.

FIGS. 1A-1D are schematic diagrams illustrating a spinal cord/brainstem-machine interface (SCMI), according to various aspects of the present disclosure.

FIGS. 2A-2D are schematic diagrams illustrating arrangements of intraspinal probes, according to various aspects of the present disclosure.

FIGS. 3A-3F are schematic diagrams illustrating different types of electrodes that can be employed on the intraspinal probes shown in FIGS. 2A-2D, according to various aspects of the present disclosure.

FIG. 4 illustrates a spinal cord/brainstem-machine interface (SCMI) system, according to various aspects of the present disclosure.

FIGS. 5A-5C are schematic and block diagrams illustrating a bi-directional, closed-loop limb control device system, according to some aspects of the present disclosure.

FIGS. 6A-6D are schematic and block diagrams of a bi-directional, closed-loop bladder control device system, according to some aspects of the present disclosure.

FIG. 7 is a flowchart illustrating a bi-directional closed-loop bladder control process, according to some aspects of the present disclosure.

FIG. 8, is a flowchart illustrating a method for a central nervous system (CNS) transfer onto a robotic body for life extension, according to various aspects of the present disclosure.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. It will be apparent, however, to those skilled in the art that these concepts may be practiced without these specific details. In some instances, well-known structures and components are shown in block diagram form in order to avoid obscuring such concepts.

As described herein, the use of the term “and/or” is intended to represent an “inclusive OR,” and the use of the term “or” is intended to represent an “exclusive OR.” As described herein, the term “exemplary” used throughout this description means “serving as an example, instance, or illustration,” and should not necessarily be construed as preferred or advantageous over other exemplary configurations. The term “coupled” used throughout this description means “connected, whether directly or indirectly through intervening connections (e.g., a switch), electrical, mechanical, or otherwise,” and is not necessarily limited to physical connections. Additionally, the connections can be such that the objects are permanently connected or releasably connected. The connections can be through switches.

Brain-Machine Interfaces (BMIs) have been in development for the last 30 years and have experienced a boom in the last decade in response to reaching significant breakthroughs. Some of these breakthroughs include the development of integrated circuits coupled with an invasive interface in the form of microneedles that record brain activity which is then interpreted and sent to a computer or actuator, which translates the patient's intent into action. Other of these breakthroughs rely on less invasive methods such as surface electrodes to deliver the same objective of delivering a transformative technology that holds significant promise in revolutionizing various fields, including neuro-prosthetics, rehabilitation, and human-computer interaction.

While these breakthroughs rely on recording brain activity (e.g., recording responses), other breakthroughs rely on stimulating a patient's brain to deliver specific therapies. Prominent examples of stimulation-driven therapy are deep brain stimulation, spinal cord stimulation for chronic pain reduction, sacral nerve stimulation to induce bladder and bowel voiding, responsive neurostimulation (RNS) which is a type of neuromodulation therapy used to treat certain types of epilepsy, vagus nerve stimulation, and the like. In short, by decoding neural activity and translating it into actionable commands, BMIs offer individuals with neurological disorders new avenues for communication, control, and restoration of lost functions.

In practice, BMIs that aim to provide effective therapies for patients with paralysis mostly target the brain. Despite impressive results, devices that rely on interaction between a neural interface and the brain to decipher the patient's intent incur significant roadblocks that are inherent to the brain's architecture. In particular, the brain's architecture involves neurons in the cortex that form an extremely complex three-dimensional network involving billions of neurons. More importantly, neural networks within the brain form interdependent connections that span several centimeters, which contributes to difficulties associate with mapping functional networks of the brain.

For instance, moving one's arm involves the interaction of neurons in the occipital, parietal, and frontal lobes as part of a coordinated activity to initiate a target task. Implanting a recording neural chip in the parietal lobe, above the motor cortex, is likely insufficient to understand when a movement is being initiated, such as the moving of one's arm (e.g., a specified limb). Additionally, any given cortical neuron synapse is often competing with thousands of other excitatory and inhibitory neuron synapses. Due to this competitive complexity, recording from any given neuron fails to provide a sufficient, absolute value signal. As a result, a relatively large body of neurons is recorded to enable deciphering of neuronal patterns that are ultimately associated with a target behavior. Consequently, forming a behavioral-neural interface match involves significant patient participation with a medical team, which is time consuming, computationally expensive, power hungry, and heavily reliant on patient compliance.

One of the primary applications of BMIs is in the development of neuro-prosthetic devices aimed at restoring motor function in individuals with paralysis or limb loss. By interfacing with prosthetic limbs or exoskeletons, BMIs enable users to control these devices with their thoughts, effectively bypassing the damaged neural pathways. Additionally, BMIs hold promise in neurological rehabilitation by providing closed-loop feedback mechanisms for promoting neuroplasticity and facilitating motor learning in individuals recovering from stroke or spinal cord injury.

Some aspects of the present disclosure are directed to an implantable, bi-directional, closed-loop intraspinal machine interface configured for implanting at the base of the brainstem or any section of the spinal cord. For example, this interface is composed of a stimulating and a sensing microelectrode array that is patterned on single or multiple shanks. In operation this interface receives inputs from sensors such as pressure/temperature that are either connected to the patient's body or are active components of a robotic body. In various aspects of the present disclosure, the sensors' stimuli are relayed to the patient's brain by means of stimulating target spinal tracts so that the patient's sensation is restored.

Various aspects of the present disclosure relate to the design and fabrication of the closed-loop, bi-directional, implantable micro-sized probe configured for implanting in the brainstem or the spinal cord and allowing bi-directional communication between synthetic actuators/sensors and the human brain. This implantable probe incorporates stimulating and sensing electrodes that are patterned on single or multiple shanks to enable selective stimulation and sensing of different areas of the human brain.

In operation, a patient's volitional motor control is restored via the sensing portion of the intraspinal probe which detects action potentials coming from the patient's brain. For example, when the patient wants to perform a movement (e.g., lift an arm) a complex signal, which often starts in the patient's prefrontal cortex, is sent to the primary and accessory motor areas (e.g., supplementary motor area (SMA), basal ganglia motor area, and/or cerebellum) and integrated in the thalamus before being sent down the motor tracts of the spinal cord. According to various aspects of the present disclosure, a disclosed probe is implanted in the spinal cord or lower brainstem. As a result, the disclosed probe captures signals originating from the thalamus and traveling through the motor tracts of the spinal cord's white matter. In this context, the location of these action potentials within the spinal cord is associated with a specific movement the patient intends to perform. By mapping these action potentials to the corresponding motor actions, it becomes possible to help patients regain voluntary control over their movements. These action potentials are detected by the sensing unit and transmitted to an external actuator that carries out a brain-initiated task.

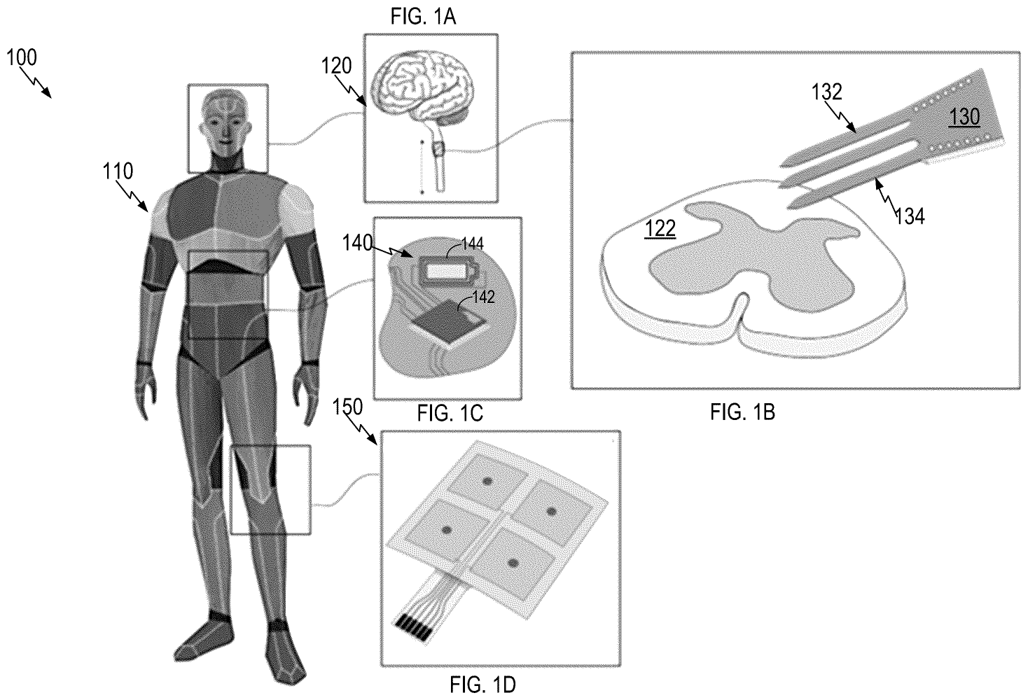

FIGS. 1A-1D are schematic diagrams illustrating a spinal cord/brainstem-machine interface (SCMI), according to various aspects of the present disclosure. As shown in FIGS. 1A-1D, various aspects of the present disclosure are directed to an SCMI 100 that is coupled with external sensors and actuators to treat several paralysis-related complications and offer significantly increase human lifespan. In some implementations, the proposed SCMI is coupled with a synthetic body and applied to patients with terminal diseases as a potential alternative to death.

FIGS. 1A-1D show a schematic representation of the SCMI 100, according to various aspects of the present disclosure. In this implementation, a synthetic body 110 is envisioned. The synthetic body 110 is equipped with sensors and actuators 150 (see FIG. 1D) that communicate with a patient's brain 120 (see FIG. 1A) to establish a bi-directional functionality (sensory and motorial). In this implementation, the patient's brain 120 (and potentially the spinal cord) is removed from the patient's biological body and inserted into the synthetic body 110. A life sustaining machine, such as a miniaturized version of an extracorporeal membrane oxygenation, can be employed for this task.

As shown in FIG. 1B, an intraspinal probe 130 is implanted at the base of the patient's brain 120 (e.g., lower brainstem) or near the cervical level 122 (e.g., levels C1/C2). In various aspects of the present disclosure, the intraspinal probe 130 includes a stimulating electrode array 132 and a sensory array 134. The stimulating electrode array 132 interfaces with afferent (sensory) axon bundles. In various aspects of the present disclosure, stimulating electrodes of the stimulating electrode array 132 interface with the afferent/sensory axon bundles so that sensation can be evoked in the patient. The stimulating electrode array are placed in the spinal cord's white matter region responsible for processing sensory information, such as the dorsal area (e.g., fasciculus cuneatus and gracilis).

In some implementations, motor axon bundles (efferent)—like the corticospinal tract—interface with the sensory array 134, which detect the patient's intention to start a given movement. The sensory portion of the sensory array 134 is placed in the spinal cord's white matter region responsible for processing motor information, such as the mediolateral (corticospinal tract) and ventrolateral (reticulospinal tract) regions of the spine. The sensory array is placed in the section of the spinal cord that is occupied by descending/efferent axon bundles which carry information about the patient's intention to carry out movement. These descending/efferent axon bundles are located in the white matter of the spinal cord. For example, a spinal tract such as the corticospinal tract is an example of a spinal tract that can be used to detect action potentials coming from the patient's motor cortex which yields information about the patient's intention to achieve specified movements.

In various aspects of the present disclosure, the intraspinal probe 130 includes a sensory array 134 that interfaces with efferent axon bundles located in the white matter of the spinal cord. For example, a spinal tract such as the corticospinal tract is an example of a spinal tract that can be used to detect action potentials coming from the patient's motor cortex which yields information about the patient's intention to achieve specified movements.

As further illustrated in FIG. 1B, the stimulating electrode array 132 of the intraspinal probe 130 interfaces with the afferent axon bundles located in the white matter of the spinal cord. In this example, spinal tracts such as the fasciculus gracilis and fasciculus cuneatus are examples of spinal tracts that can evoke sensation in the patient's brain via the stimulating electrode array 132 of the intraspinal probe 130. According to various aspects of the present disclosure, the stimulating electrode array 132 is composed of a three-dimensional (3D) electrode array, including a set of nanopatterned electrodes. In some implementations, each electrode of the stimulating electrode array 132 is composed of a black silicon (BSi) of varying shapes (or a predetermined shape), coated with a biocompatible metal such as Platinum, Iridium Oxide, Iridium, or Gold. The different BSi features can take different aspect ratios (e.g., ranging from 2:1 to 100:1).

As shown in FIG. 1C, an application specific integrated circuit (ASIC) module 142 and a power module 144 are housed in a sealed, conductive enclosure 140, configured for insertion in the synthetic body 110. The ASIC module 142 is responsible for coordinating the bi-directional, closed-loop communication between the synthetic body 110 and the brain 120.

Conventional brain-machine interfaces (BMIs) exhibit the limitations of: (1) lack of bi-directionality; (2) lack of closed-loop functionality; (3) significant power consumption specification; and (4) lack of scalability. For example, regarding the lack of bi-directionality, conventionally available BMIs either record or stimulate neural tissue. For instance, an ideal neural-prosthetic limb would allow the patient to both move the limb and feel stimuli that are being applied to the neural-prosthetic limb. Currently, this neural-prosthetic limb is not possible with conventional BMIs and partially possible with interfaces that are based on interacting with peripheral nerves, which have their own limitations especially related to poor resolution.

Additionally, the inability to provide bi-directionality in conventional BMIs is closely related to their lack of providing closed-loop therapy. Nature is intrinsically governed by feedback loops that drive one's cause-consequence behaviors. Additionally, physical stimuli such as pressure, heat, visual, and auditory are specified for one's brain to initiate a target action. For example, deep brain stimulation (DBS) allows for dampening of a patient's tremors by the patient's triggering of the device. DBS is currently incapable of understanding when a tremor should be stopped solely based on brain activity because DBS lacks the ability to sense brain activity. Similarly, other solutions allow the patient to move a cursor on a computer's monitor, but are unable to stimulate the patient's brain and, therefore, are unable to restore the patient's sensation.

In practice, brain networks are composed of millions of neurons that connect over a large spatial frame (e.g., several centimeters). Consequently, complex algorithms are necessary to decipher what a given network is trying to achieve. This task is achieved by collecting large amounts of data that is recorded at a high sampling rate over a large number of electrodes (e.g., >1000) leading to excessive data rates (e.g., exceeding 1 Gbps). Such high data rates consume significant amounts of power, which can heat up living tissues and, potentially, result in tissue damage.

Furthermore, conventional BMIs aim to solve a specific, functionality constrained problem, such as restoring speech, hearing, vision and the like. Unfortunately, current BMIs are inadequate for restoring several physical functionalities at the same time. For instance, restoring mobility in a paraplegic patient would involve a brain interface that spans the majority of the motor cortex, which occupies tens of square centimeters. Such a large device would potentially cause massive brain inflammation, heating and swelling, which could lead to serious medical concerns. In short, current BMIs lack the ability to: (1) restore multiple functionalities in paralyzed patients; (2) function in closed-loop modality; (3) restore sensation; and (4) provide scalability to cover several areas of the human brain.

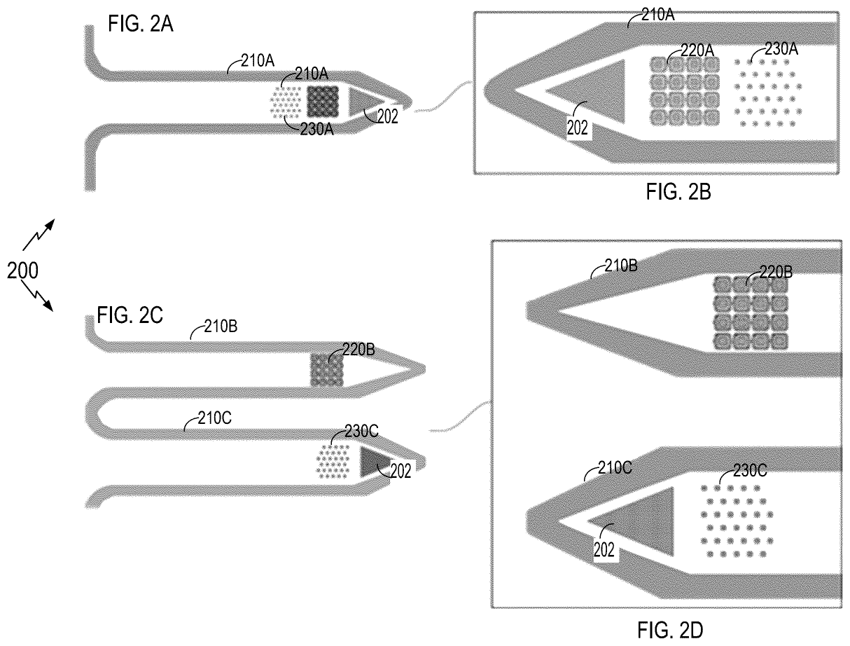

FIGS. 2A-2D are schematic diagrams illustrating arrangements of intraspinal probes, according to various aspects of the present disclosure. In various aspects of the present disclosure, FIGS. 2A-2D illustrate schematic implementations of intraspinal probes, such as shown in FIGS. 1A-1D. As shown in FIGS. 2A and 2C, an intraspinal probe 200 can be patterned to have a single protruding, implantable shank 210A, as shown in FIG. 2A, or multiple protruding, implantable shanks 210B and 210C, as shown in FIG. 2C. Each implementation of the shanks 210 includes a reference electrode array 202.

As further illustrated in FIGS. 2B and 2C, the shanks 210 are composed of a semiconductor material (e.g., bulk silicon (Si)) having a predetermined thickness (e.g., ranging from 10-100 microns). Additionally, the widths of the shanks 210 vary based on the number of electrodes housed on the shanks 210. Each shank may vary according to a predetermined width (e.g., 50-800 microns) and a predetermined inter-shank spacing (e.g., 100 microns). In this example, a length of each of the shanks 210 varies according to a predetermined range (e.g., 2-15 millimeters). In this implementation, a stimulating electrode arrays 220A and sensing electrode arrays 230A can be arranged on the single protruding, implantable shank 210A, where the stimulating electrode array 220A is shown on top of the sensing electrode arrays 230A. In operation, an action potential is detected from the sensing electrode arrays 230A, relative to the reference electrode array 202.

The multi-shank configuration shown in FIG. 2C may be useful in certain situations in which the sensing and stimulating tracts are far separated from one another by a significant distance. For example, sensation of the foot at the cervical level can be restored by stimulating the medial section of the dorsal column (i.e., a medial section of the fasciculus gracilis). The efferent tract for the same body part (i.e., foot) is located in the lateral section of the corticospinal tract. In humans the distance between these two tracts is on the order of a few millimeters. Therefore, using two thin shanks of the multi-shank configuration shown in FIG. 2C, instead of large one shank would cause less tissue damage upon implantation. Conversely, the sensory and motor tracts for micturition are relatively close and positioned on top of each other so a signal shank may be used without causing extra damage.

As shown in FIG. 2D, the stimulating and sensing electrode arrays can be arranged on multiple shanks, in which the stimulating electrode array 220B is shown on the right shank 210B, while the sensing electrode arrays 230C are shown on the left shank 210C. In some implementations, this arrangement of the multiple protruding shanks 210B, 210C of the intraspinal probe 200 is utilized in situations where the target area for sensing action potentials is located a significant distance (e.g., >1 mm) from the target area for stimulation.

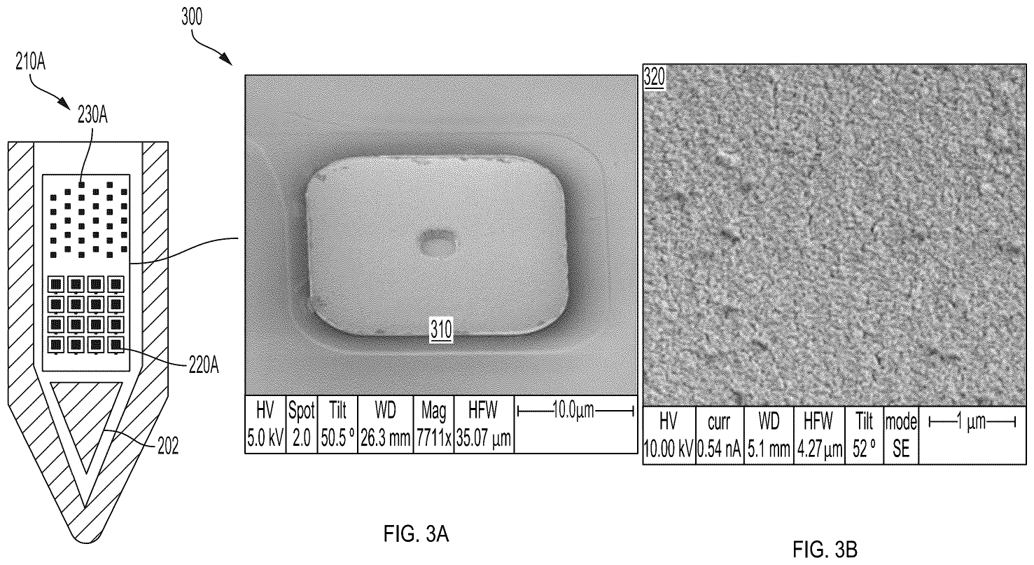

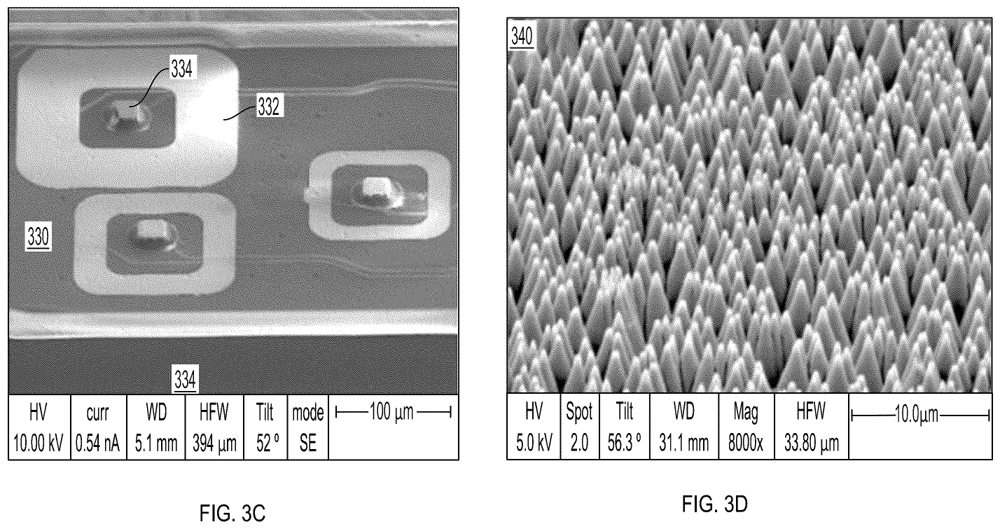

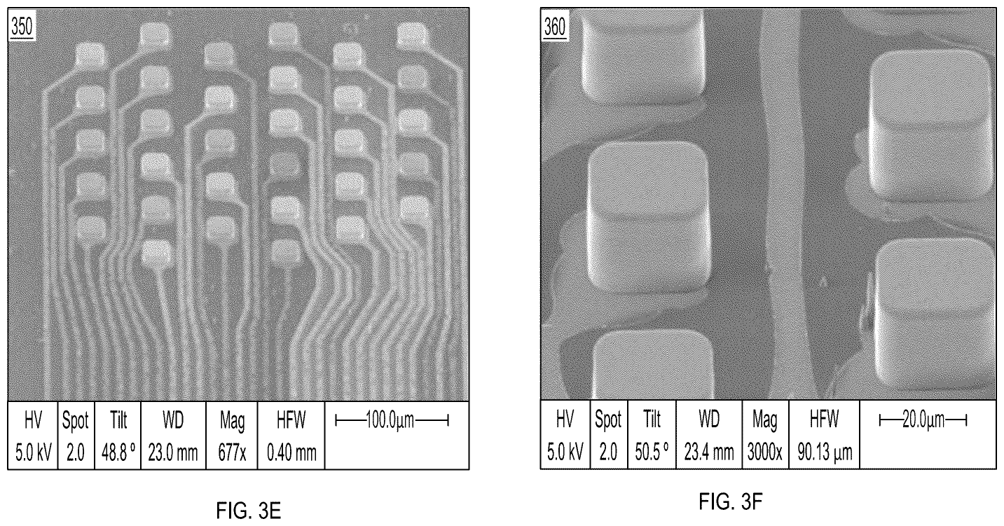

FIGS. 3A-3F are schematic diagrams illustrating different types of electrodes that can be employed on intraspinal probes, for example, as shown in FIGS. 2A-2D, according to various aspects of the present disclosure.

FIGS. 3A-3F illustrate various types of electrodes that may be employed by an intraspinal probe, for example, as shown in FIGS. 2A-2D. The single protruding, implantable shank 210A of FIG. 2A is provided as an example, intraspinal probe. For example, the stimulating electrode array 220A can be scaled up to 500 electrodes per mm² which allows the selective stimulation of thousands of electrogenic cells (such as neurons or cardiomyocytes) in parallel which is essential in furthering the study of neuronal networks. The stimulating electrode array 220A can achieve high resolution (<80 μm in lateral current spread) neural stimulation, which enables high spatial selectivity (<100 μm).

As shown in FIG. 3A, the sensing electrode arrays 230A may be implemented utilizing a two-dimensional (2D) flat electrode 310. In this example, the single electrodes (e.g., the 2D flat electrode 310) of the sensing electrode arrays 230A are arranged in a 2D matrix in a first direction and a second direction. The electrodes are controlled according to a design layout and are easily customizable. In this example, the 2D flat electrode 310 is coated with a conductive material (e.g., high roughness platinum (Pt)) and configured for sensing and/or stimulating.

According to various aspects of the present disclosure, a high-pressure sputtering is performed to deposit the conductive material (e.g., Pt) to reach a predetermined level of roughness, as shown by a conductive material surface 320 in FIG. 3B. In practice, formation of the 2D flat electrode 310 utilizing the conductive material surface 320 yields a lower impedance relative to conventional flat, smooth electrodes of the same dimensions due to the larger effective surface area (e.g. ˜60 kΩ at 1 kHz for high roughness Pt-coated electrodes as opposed to ˜300 kΩ at 1 kHz for smooth Pt-coated electrodes of the same size, 35×35 μm²). The lower impedance beneficially reduces noise, which enables sensing of smaller signals. Additionally, the conductive material surface 320 increases the effective electrode surface area, which increases the charge delivery capacity of such electrodes, such as the 2D flat electrode 310 in case such electrodes are used for stimulation.

In various aspects of the present disclosure, nano-patterned electrodes are utilized to improve signal to noise ratio (SNR) and effective charge delivery. Such nano-patterned electrodes can be used in place of the 2D flat electrode 310, which are utilized for sensing. FIG. 3C illustrates a scanning electron microscopy image of black-silicon-based nanopatterned, three-dimensional (3D) electrodes 330, including a return electrode 332 surrounding a stimulating electrode 334, which are utilized for stimulation. In this example, the 3D electrodes 330 are coated with a conductive material (e.g., platinum (Pt), titanium nitride (TiN), gold (Au), iridium (Ir), or iridium oxide.

In some implementations, the 3D electrodes 330 may be formed from the 2D flat electrode 310 by depositing a blanket layer of amorphous silicon (α-Si) on the wafer and subsequently patterning such blanket layer to form black silicon (Bsi) on the entire surface of the 2D flat electrode 310. Silicon cryo-etching is performed (SF₆/O₂,) in which oxygen condensation at very low temperatures (e.g. −130° C.) forms a micro-masking layer which yields dense, high aspect ratio silicon features (e.g., silicon grass), such as a needle-like morphology 340 shown in FIG. 3D. Next, an etching process is performed to separate the stimulating electrode 334 (e.g., composed of BSi grass) from the surrounding, return electrode 332. As shown in FIG. 3C, the BSi-based 3D electrodes 330 exhibit a 3D shape.

In this example, the BSi-based 3D electrodes 330 are coated in conductive material such as titanium (Ti)/gold (Au), Ti/platinum (Pt), Ti/Pt iridium (Ir), Ti/Pt/Ir, Ti/Pt/Ir oxide (IrOx), Ti/Ir, Ti/Ti nitride (N), Ti/Pt/TIN, Ti/IrOx. The BSi-based 3D electrodes 330 may be implemented using a low temperature poly silicon deposition that is compatible with complementary metal oxide semiconductor (CMOS) technology.

The BSi-based 3D electrodes 330 are configured to achieve an electrical impedance (e.g., <50 kΩ at 1 kHz) for a geometric area (e.g., 20×20 μm²), which allows for low voltage evolution (e.g., −0.6V<Vₛₜᵢₘᵤₗₐₜᵢₒₙ<0.8V) during stimulation. The BSi-based 3D electrodes 330 are able to increase the total 3D electrode surface area (e.g., from 2-10 times), thereby allowing for a drastic increase in charge density capacity (>1 mC/cm²). The BSi-based 3D electrodes 330 are able to limit biofouling over time, which limits the electrode increase in impedance to a maximum of 1 order of magnitude, for example, from a pristine impedance of 30 kΩ to an impedance of 300 kΩ at 1 kHz after one month of implantation.

As shown in FIG. 3D, the needle-like morphology 340 composed of black silicon (BSi) is used to form the stimulating electrode 334 and/or the return electrode 332, which allows for an exponential increase in the effective surface area of the stimulating electrode 334 by providing a third dimension, which drastically decreases the electrode impedance and increases the electrode charge delivery capacity. This added third dimension provided by the needle-like morphology 340 of the BSi-based 3D electrodes 330 supports closer contact between the stimulating electrode 334 and target axons, which improves the SNR. Additionally, lower impedance improves stimulation by decreasing the voltage that develops at the electrode/tissue interface during stimulation, which is safer for the patient.

FIGS. 3E and 3F illustrate an alternative to the BSi-based nanopatterning, which involves the fabrication of micro-post electrodes 350, which are shown in an exploded view 360 in FIG. 3F. In some implementations, the micro-post electrodes 350 can be coated in conductive materials (e.g., Pt, TiN, Au, Ir, IrOx) to provide a conductive region surrounding an electrode post (e.g., the micro-post electrodes 350). Similarly to the BSi-based electrodes, the micro-post electrodes 350 decrease the distance between the target axons and the micro-post electrodes 350, which allows for more effective stimulation and sensing.

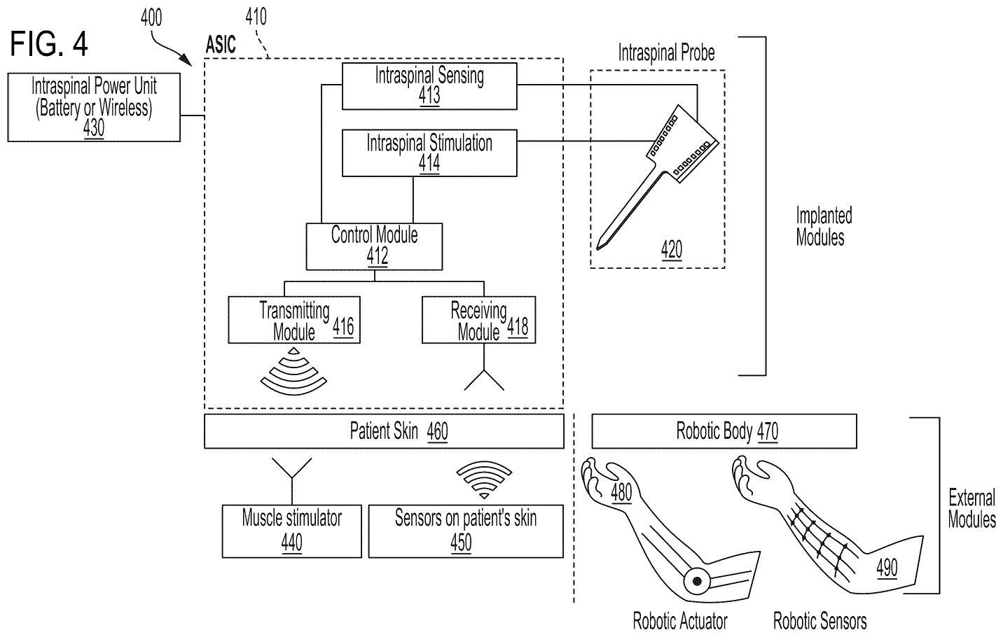

FIG. 4 illustrates a spinal cord/brainstem-machine interface (SCMI) system, according to various aspects of the present disclosure. As shown in FIG. 4, the SCMI system 400 is composed of an application specific integrated circuit (ASIC) 410, an intraspinal/brainstem sensing and stimulating probe (intraspinal probe 420), a power source 430, an external actuator/muscle stimulator 440, and an external sensor 450. In some implementations, the power source 430 is implemented using a battery or, alternatively, the power source 430 is implemented utilizing wireless communication. In this example, the external actuator/muscle stimulator 440 and/or the external sensor 450 allow for communication between the intraspinal probe 420, the ASIC 410, and the outside world. The external actuator/muscle stimulator 440 and/or the external sensor 450 may be implemented as any device/sensor/machine/actuator that is housed outside the patient's body. For example, this communication is accomplished through biocompatible, stress-free cables (e.g., silicone-coated platinum wires) or wirelessly.

As shown in FIG. 4, the SCMI system 400 utilizes the intraspinal probe 420, which is shown in a single shank configuration of the intraspinal probe 200 shown in FIGS. 2A-2D. For example, as shown in FIGS. 2A and 2B, the shanks 210 are composed of a semiconductor material (e.g., bulk silicon (Si)) having a predetermined thickness (e.g., ranging from 10-100 microns). Additionally, the widths of the shanks 210 vary based on the number of the electrodes housed on the shanks 210. The sensing and stimulating electrodes vary in size (e.g., ranging from 5×5 μm² to 35×35 μm²) and are coated in biocompatible and conductive materials (e.g., Pt, TiN, Au, Ir, IrOx). The sensing and stimulating electrodes may be implemented utilizing flat or nanopatterned electrodes. For example, nanopatterned electrodes may be implemented utilizing a black silicon (BSi) technique or a nano/micro-pillar technique, as described in co-pending U.S. patent application Ser. No. 18/932,426.

In some implementations, the ASIC 410 that drives the SCMI system 400 is integrated in the base of the intraspinal probe 420, which is defined as the section of the intraspinal probe 420 that houses bonding pads. Alternatively, the ASIC 410 is implemented as a free-standing chip which can be housed in a sealed can and implanted subcutaneously in a similar fashion to devices, such as the pacemakers or deep brain stimulators.

As shown in FIG. 4, the ASIC 410 includes a control module 412, which coordinates the functions of the SCMI system 400. For example, the control module 412 manages the timing (clocks) of the SCMI system 400, enables either an intraspinal sensing module 413 or an intraspinal stimulation module 414, maps and thresholds action potentials, and enables a transmitting module 416 and a receiving module 418. In this example, the intraspinal stimulation module 414 is implemented using a current controlled stimulator configured to deliver balanced, bi-phasic, or monophasic current pulses with a predetermined amplitude (e.g., ranging from 20-100 microamperes (μA)).

Additionally, the intraspinal stimulation module 414 utilizes a dual-frequency scheme to deliver effective stimulation pulses. In some implementations, a carrier frequency defines the stimulation on/off time and is configured with a predetermined carrier frequency range (e.g., from 1-50 Hz). In some implementations, a second frequency which is configured with a predetermined stimulating frequency range (e.g., from 50-200 Hz) and is turned on during the rising edge of the carrier frequency. A duration of the stimulation train is configured according to predetermined ranges (e.g., from 50-800 milliseconds (ms)). Each stimulating pulse ranges in duration from, for example, 100-400 microseconds (μs) per phase.

As further illustrated in FIG. 4, the intraspinal sensing module 413 is implemented using an array of action potential detectors. In some implementations, each action potential detector is composed of a low noise amplifier followed by a programmable gain amplifier (PGA), which can be shared among different channels or can have a dedicated single channel. In operation, the PGA output feeds into an analog-to-digital convertor (ADC) which thresholds neural signals. The ADC can take the form of a 1-bit comparator that thresholds the incoming neural activity and outputs a digital 1 or 0 when the threshold is reached, effectively enabling transmission of action potentials for further processing.

For example, the action potentials are transmitted from the intraspinal sensing module 413 to the control module 412, which maps and analyzes the action potential signals. In some implementations, action potentials that are received at a given frequency and last for a predetermined amount of time are relayed to the transmitting module 416. The transmitting module 416 may employ a microfabricated radio frequency (RF) antenna to relay the information coming from the control module 412 to the external actuator/muscle stimulator 440. Additionally, the receiving module 418 is implemented using a microfabricated receiving RF antenna coupled to a low noise amplifier which receives information from the external sensor 450 and relays this information to the control module 412.

In some implementations, the ASIC 410 is powered using the power source 430, which may be composed of a battery or configured with wireless charging functionality. The ASIC 410 and the power source 430 may be housed in a titanium or titanium alloy, which is implanted subcutaneously. Additionally, the ASIC 410 may be connected to the intraspinal probe 420, via flexible cables or wirelessly. In various aspects of the present disclosure, a maximum power of the ASIC 410 is maintained under a predetermined level (e.g., 50 mW). This predetermined level is selected based on an established biological safety limit of 1 degree Celsius (1° C.) to avoid possible tissue heating and damage to a patient. In this example, the external sensor 450 and the external actuator/muscle stimulator 440 communicate with the ASIC 410 using RF links once the ASIC 410 is implanted in a patient's skin 460.

In some implementations, the ASIC 410 is configured to detect action potentials originating from the efferent portion of the patient's spinal cord using the sensing array (e.g., the intraspinal sensing module 413 from the intraspinal probe 420) and to stimulate the afferent portion of the patient's spinal cord using the stimulating electrode array (e.g., the intraspinal stimulation module 414 through the intraspinal probe 420), thereby restoring the sensation of pain, temperature, and pressure. In various aspects of the present disclosure, a functional map linking specific regions of the spinal cord to their respective sensory and motor functions is generated through the combined sensing and stimulation capabilities of the array (e.g., the intraspinal probe). In some implementations, this map enables the system to interpret the patient's motor intentions and selectively stimulate sensory responses corresponding to different areas of the body.

As further illustrated in FIG. 4, the SCMI system 400 restores sensation and full body motion in quadriplegics or could allow for communication between an extracted brain and a robotic body 470 to extend the life of a terminally ill patient using the noted functional map. In particular, various aspects of the present disclosure are directed to central nervous system (CNS) or brain transfer into a synthetic body 110, as shown in FIG. 1, for example using a robotic actuator 480 and/or robotic sensors 490 of the robotic body 470. In some implementation, the SCMI system 400 is utilized to communicate bi-directionally with the robotic body 470, which is provided to represent a patient's artificial body.

For example, a terminally ill patient that has no alternatives to death might opt for brain central nervous system (brain and spinal cord) or brain only extraction and insertion into the robotic body. In this implementation, SCMI system 400 is implanted at the base of the patient's brainstem for supporting back and forth communication between the patient's brain and the robotic body 470. Other implementations involve movement and sensory restoration in paraplegic and quadriplegic patients. In this example, the SCMI system 400 can sense and actuate the patient's body or the robotic actuator 480 and/or the robotic sensors 490 of the robotic body 470.

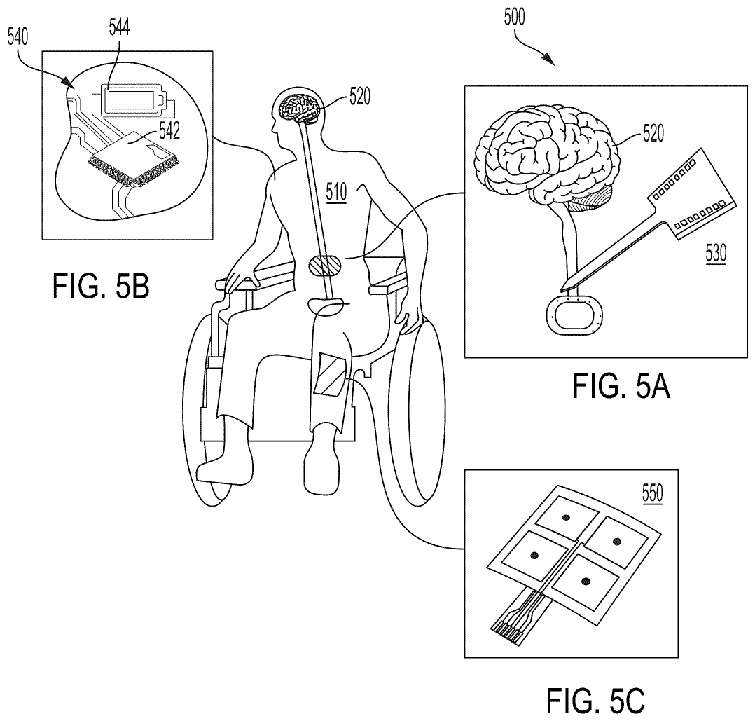

FIGS. 5A-5C are schematic and block diagrams illustrating a bi-directional, closed-loop limb control device system 500, according to some aspects of the present disclosure. In this implementation, the bi-directional, closed-loop limb control device system 500 includes a patient 510 equipped with sensors and actuators 550 (sec FIG. 5C) on a lower leg that communicate with a patient's brain 520 (see FIG. 5A) to establish a bi-directional functionality (sensory and motorial). In this implementation, an intraspinal probe 530 is implanted within the spine of the patient 510 to interface with the white matter of the spinal cord. As shown in FIG. 5B, an application specific integrated circuit (ASIC) module 542 and a power module 544 are housed in a sealed, conductive enclosure 540, configured for insertion in the patient 510. The ASIC module 542 is configured to coordinate the bi-directional, closed-loop communication between the sensors and actuators 550 and the patient's brain 520 using the intraspinal probe 530.

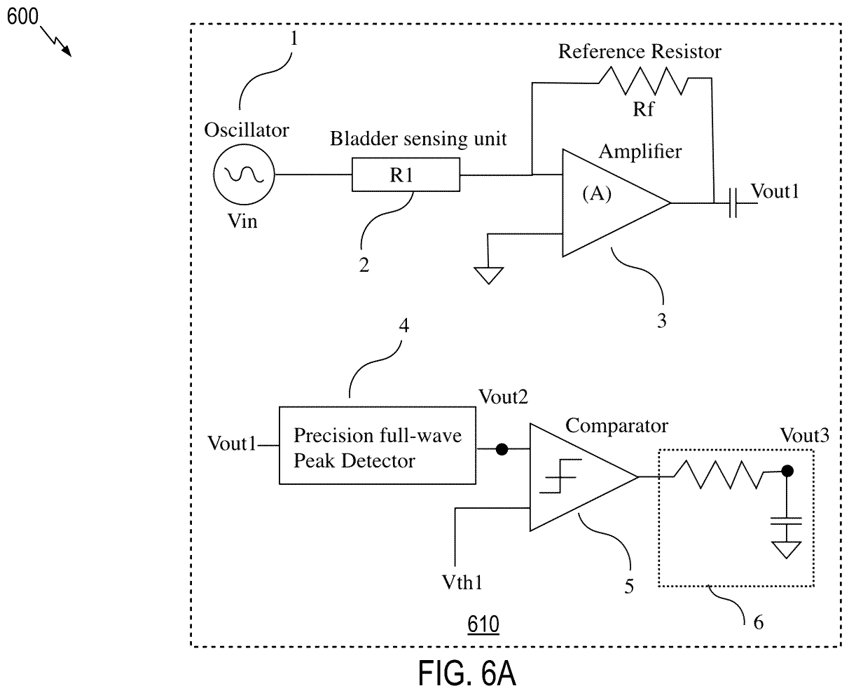

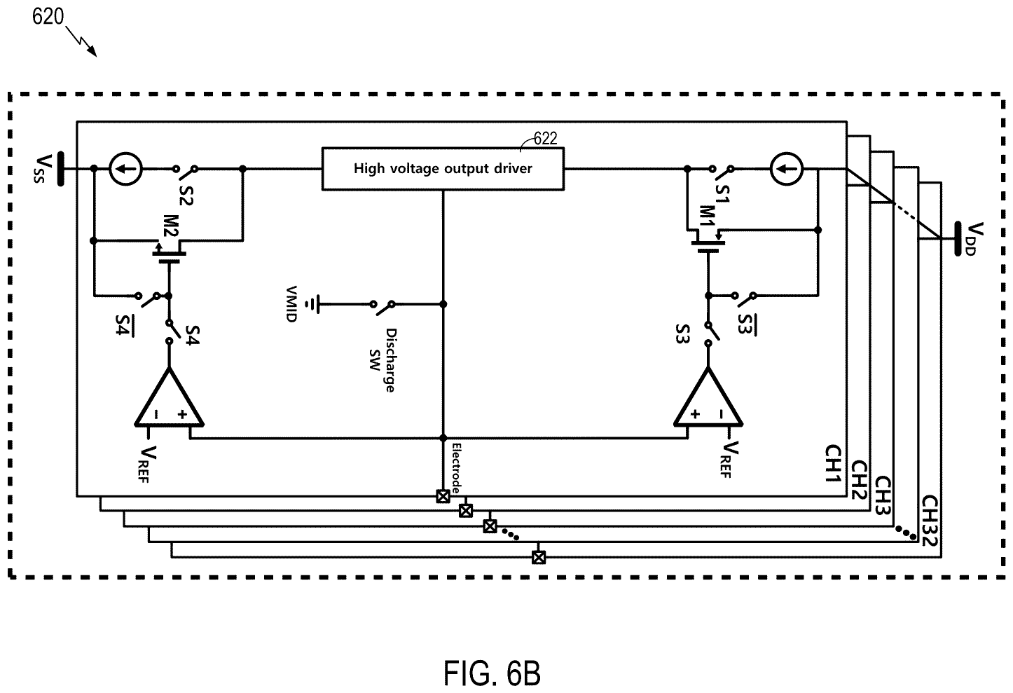

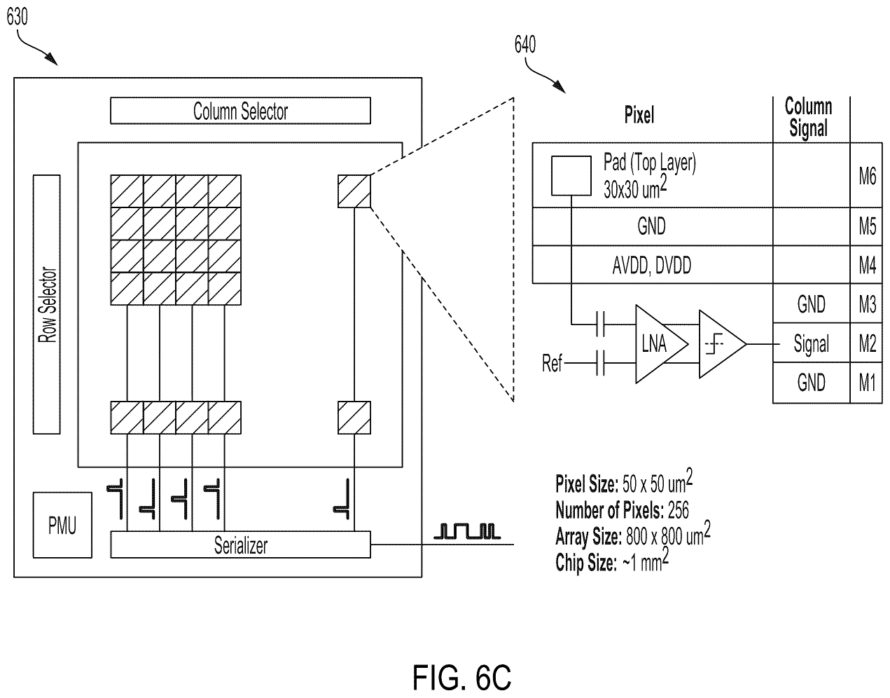

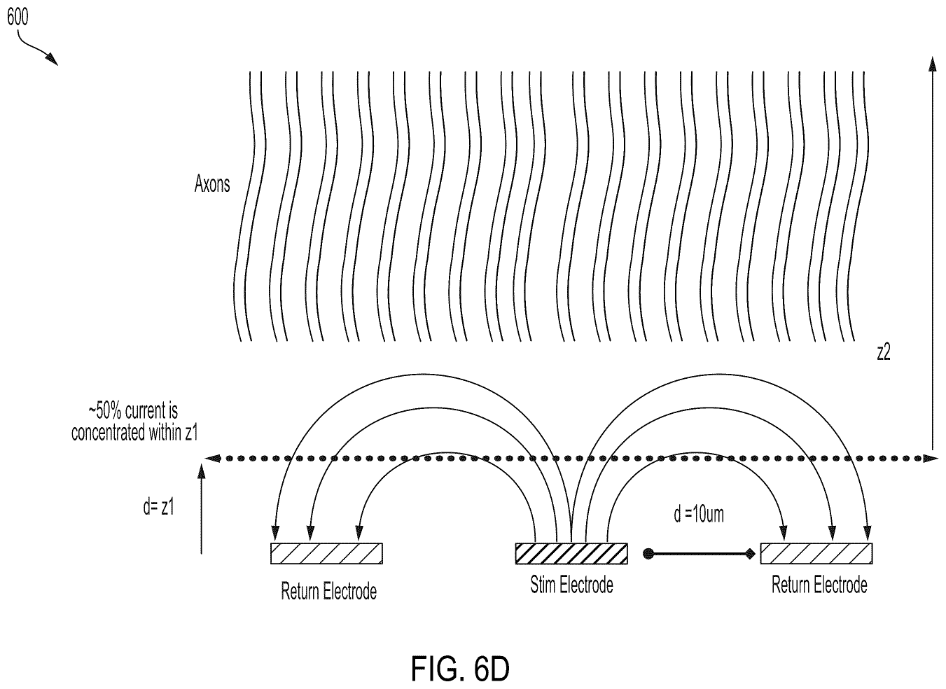

FIGS. 6A-6D are schematic and block diagrams of a bi-directional, closed-loop bladder control device system 600, according to some aspects of the present disclosure.

FIG. 6A is a schematic diagram illustrating a bladder monitor 610, according to various aspects of the present disclosure. As shown in FIG. 6A, the bladder monitor 610 includes a voltage-controlled pattern generator (e.g., oscillator [1]) that produces a sine wave at a given frequency and input voltage Vin. For instance, the sine wave generated by the oscillator [1] has a frequency of 1 kHz and a Vin of 100 mV peak-to-peak. Additionally, the bladder monitor 610 includes a bladder sensing unit (BSU) [2], coupled between the oscillator [1] and an amplifier [3]. In operation, a current travels from the oscillator [1] to the amplifier [3]. This current is modulated by the impedance of the BSU [2], which changes as a function of bladder volume.

In some implementations, the BSU [2] is composed of microelectrodes that are implanted on a bladder muscle (e.g., a detrusor). Two microelectrodes and the detrusor muscle form a resistive element (R1). As the bladder fills up with urine, the effective path that the current takes from microelectrode 1 to microelectrode 2 increases, which changes the net impedance of the resistive element (R1). The ratio between a reference resistor Rf and the resistive element R1 (Rf/R1) determines the gain (A) of the amplifier

. The amount of urine in the bladder modulates R1 resulting in a change of the gain (A) of the amplifier [3]. This gain change causes a change in a first output voltage (Vout1) of the amplifier [3]. For example, as the bladder fills, the resistive element R1 increases, leading to a decrease in the gain (A) which causes a decrease in the first output voltage Vout1. In this example, the first output voltage Vout1 of the bladder monitor 610 correlates to the volume of urine that is present in the bladder.

As further illustrated in FIG. 6A, a peak-to-peak value of the first output voltage Vout1 is measured by a voltage peak detector [4], which rectifies the first output voltage Vout1 and outputs the peak-to-peak full wave voltage (second output voltage Vout2). The first output voltage Vout1 is inversely related to the bladder volume. For example, the first output voltage Vout1 is at a maximum value when the bladder is empty and decreases as the urine volume increases. The second output voltage Vout2 is connected to a comparator [5] that triggers when the second output voltage Vout2 is lower than a programmable threshold Vth1. In this implementation, the bladder monitor 610 further includes a filter [6]. In some implementations, the filter [6] prevents false detection of bladder fullness that could be caused by a sudden drop in the first output voltage Vout1, such as during coughing or an abrupt movement. In this example, the filter [6] is configured to confirm that the second output voltage Vout2 remains below the threshold Vth1 for a programmable, continuous amount of time before the signal can be interpreted as “bladder full” (e.g., based on a third output voltage Vout3).

FIG. 6B illustrates an example of a circuit that can be used as a multi-channel, stimulating array intraspinal probe for use in the bi-directional, closed-loop bladder control device system 600, according to various aspects of the present disclosure. In this example, a multi-channel, stimulating circuit 620 may be configured with thirty-two (32) stimulating channels, each including a high voltage output driver coupled between a first switch S1 and a second switch S2. The first switch S1 is coupled in series with a current source, which is in parallel with a transistor M1. The transistor M1 is coupled to a first operational amplifier through third switches S3. Similarly, the second switch S2 is coupled in series with a current source, which is in parallel with a transistor M2. The transistor M2 is coupled to a second operational amplifier through fourth switches S4. In this example, the first and second operational amplifiers have a cross-coupled positive terminal (+) and a reference voltage V_{REF} at a negative terminal (−). Additionally, an electrode is coupled to the positive terminal+of the first and second operational amplifiers and a high voltage output driver 622 through a discharge switch SW.

FIG. 6C illustrates a sensing array intraspinal probe 630 for use in the bi-directional, closed-loop bladder control device system 600, according to various aspects of the present disclosure. In this example, the sensing array intraspinal probe 630 includes an array of electrodes between a column selector and a row selector. Additionally, the sensing array intraspinal probe 630 includes a power management unit (PMU) and a serializer coupled to the array of electrodes. An exploded view of an electrode 640 illustrates a pad coupled to a low noise amplifier (LNA) that is coupled to a comparator.

FIG. 6D illustrates operation of the multi-channel, stimulating circuit 620, which uses a stimulation (stim) electrode and return electrodes for sending current pulses to axons of an afferent micturition center in response to the bladder full signal, which leads the patient to feel the urge to urinate. The stimulating pulses sent through the stimulating electrodes can be programmed for transmission at different frequencies and durations. For example, the stimulating pulses can be programmed to be biphasic or monophasic and last for a predetermined period (e.g., 100-400 μs) per phase. Additionally, the stimulating frequency can be programmed to deliver current at single (e.g., 1-100 Hz) or dual frequencies. In a dual-frequency mode, a low frequency carrier wave (e.g., 1 Hz) is combined with a stimulating frequency (e.g., 90 Hz). The high frequency stimulation may be applied for twenty percent (20%) of each cycle of low frequency carrier wave, maintaining a fixed duty cycle. Additionally, the BSU [2] (e.g., impedance monitor) is turned off while the stimulation is performed.

In operation, the stimulation lasts for a pre-programmed amount of time which can range from 10-60 seconds. When the stimulation ends, the sensing array of the sensing array intraspinal probe 630 turns on and the intraspinal stimulator of the multi-channel, stimulating circuit 620 turns off. If any of the electrodes (e.g., 16-32 electrodes) on the sensing array detects bursts of action potentials traveling down the efferent/motor micturition tract for a pre-programmed amount of time, a signal indicating that the patient intends to urinate will be relayed to a sacral nerve stimulator.

In practice, the sensing portion of the sensing array intraspinal probe 630 is inserted in the efferent/motor micturition tract and any time an action potential is detected, the comparator is triggered. When a pre-programmed number of triggered events is collected, a signal that enables a sacral anterior root stimulator (SARS) is sent. Once the SARS turns on, the sensing portion of the sensing array intraspinal probe 630 turns off. The SARS sends stimulating pulses that lead to bladder contraction and voiding.

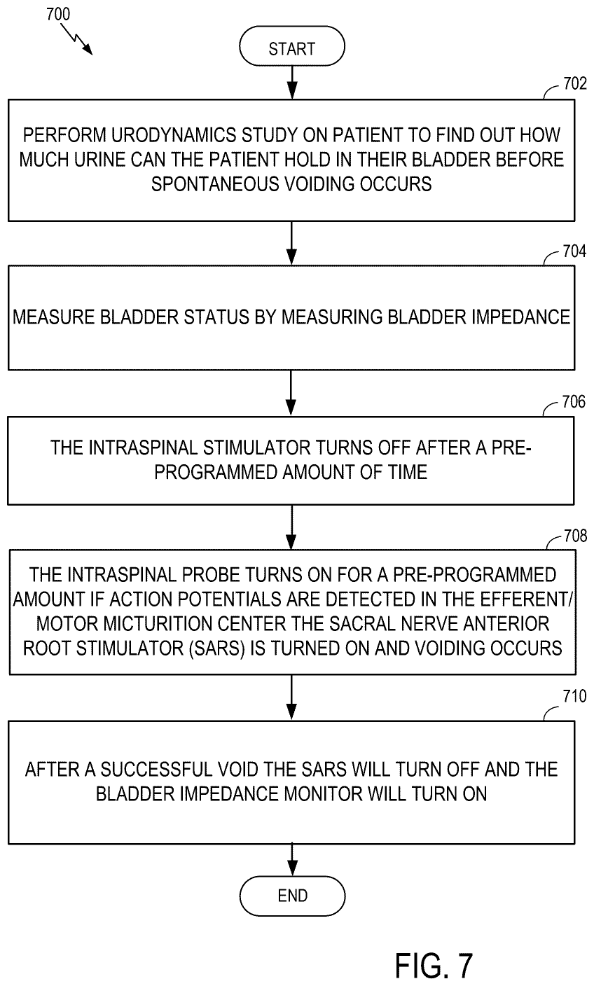

FIG. 7 is a flowchart illustrating a bi-directional closed-loop bladder control process 700, according to some aspects of the present disclosure. The bi-directional closed-loop bladder control process 700 begins at block 702, in which an urodynamics study is performed on a patient to find out how much urine the patient can hold in their bladder before spontaneous voiding occurs. At block 704, a bladder impedance is measured to determine bladder status. At block 706, once the bladder volume reaches a predetermined percentage (e.g., 70%) of the max volume that the patient can hold, the impedance monitor turns off and the stimulating portion of the intraspinal probe turns on and the patient feels the urge to urinate. During this process, the stimulating portion of the intraspinal probe turns off after a pre-programmed amount of time, such as 20-60 seconds.

At block 708, when the intraspinal stimulator turns off at block 706, the sensing portion of the intraspinal probe turns on for a pre-programmed amount of time (e.g., 10 minutes). If the patient intends to urinate, action potentials are detected in the efferent/motor micturition center for a pre-programmed amount of time (e.g., 5-15 seconds). In response, the sacral nerve anterior root stimulator (SARS) is turned on and voiding of the bladder occurs. At block 710, after a successful void of the bladder, the SARS turns off and the bladder impedance monitor is turned on. If the sensing portion of the intraspinal probe doesn't detect action potentials, the sensing portion turns off and blocks 704 to 710 are repeated. In some instances of the disclosure the bladder control device is used in sensation mode only where only the bladder sensing unit (BSU) and the intraspinal stimulator are employed. In such case when the BSU detects that a pre-defined threshold has been reached the BSU turns off and the intraspinal stimulator turns on for a pre-defined time (e.g. 1 minute) to warn the patient that their bladder needs to be emptied so that incontinence can be avoided.

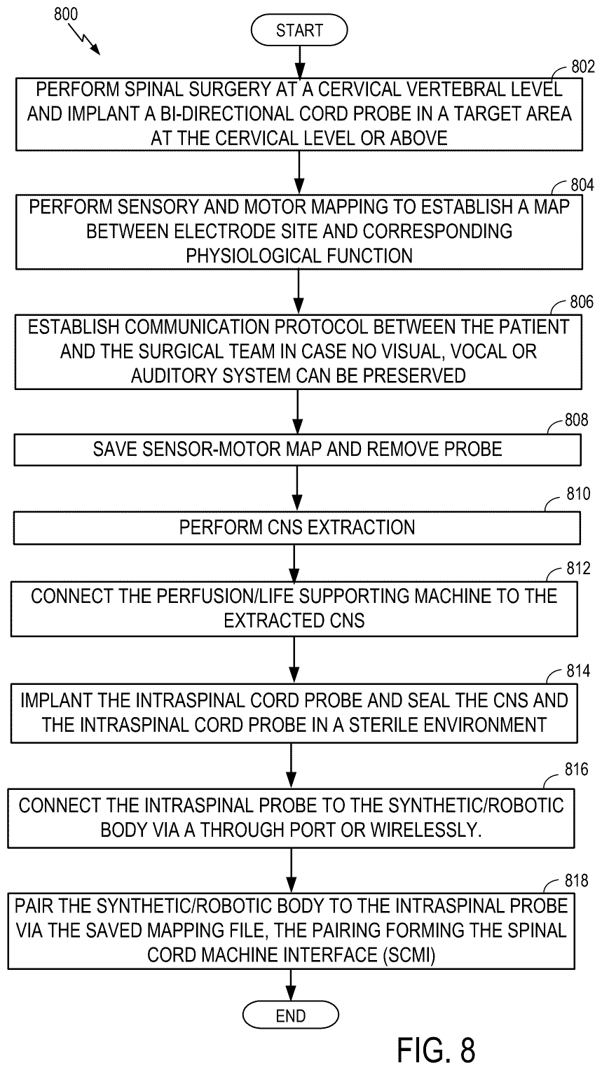

FIG. 8, is a flowchart illustrating a method 800 for a central nervous system (CNS) transfer onto a robotic body for life extension, according to various aspects of the present disclosure. As shown in FIGS. 1A-1D, the SCMI 100 is utilized to communicate bi-directionally with the patient's artificial body (e.g., the synthetic body 110). For instance, terminally ill patients that have no alternatives to death might opt for CNS or brain extraction and insertion process into a robotic body (e.g., the synthetic body 110). According to various aspects of the present disclosure, the intraspinal probe 130 is implanted at the base of the patient's brain 120 at or near the cervical level 122 of the lower brainstem. In this example, the intraspinal probe 130 enables back and forth communication between the patient's brain 120 and the robotic body (e.g., the synthetic body 110). Other applications could involve movement and sensory restoration in paraplegic and quadriplegic patients.

The method begins at process block 802, in which spinal surgery is performed at the cervical vertebrae level (e.g., C1-C7), such as the C1 cervical vertebral level. Additionally, performing spinal surgery at process block 802 also includes implanting a bi-directional cord probe in a target area at the cervical vertebrae level of a patient during an initial implant. For example, as shown in FIG. 1B, As shown in FIG. 1B, an intraspinal probe 130 is implanted at the base of the patient's brain 120 at or near the cervical level 122 of the lower brainstem or at C1 cervical vertebrae level. In various aspects of the present disclosure, the intraspinal probe 130 includes a stimulating electrode array 132 that interfaces with afferent axon bundles located in the white matter of the spinal cord. For example, a spinal tract such as the fasciculus gracilis is an example of an afferent tract that can be stimulated to evoke sensation in the patient. Another example is the corticospinal tract that can be used to detect action potentials coming from the patient's motor cortex which yields information about the patient's intention to achieve specified movements.

At process block 804, sensory and motor mapping are performed to establish a map between electrode site and corresponding physiological function. In this example, motor mapping is performed by asking the patient to move a specified body part. The patient's intent to move a specific limb is recorded by the electrode array that is placed in the spinal motor tracts such as the corticospinal tract. Motor mapping is repeated for all the muscles in the body. Sensory mapping involves asking the patient which feeling they feel upon turning on each stimulating electrode in the fasciculus cuneatus and fasciculus gracilis. Sensory mapping is rastered across the body. Two-point discrimination is used to determine the points that will be mapped.

At process block 806, a communication protocol is established between the patient and the surgical team in case no visual, vocal, or auditory system can be preserved. An example of a communication protocol involves tapping-based Morse-code. For instance, short and long stimulation of a specific area of the body provoked sensation of short and long pressure over that specified area. The short and long pulses can encode the Morse code. The patient can respond by imagining tapping their finger with for a short or long time. These events lead to action potentials in the spinal motor area related to the specified finger which are captured by the sensory section of the probe.

At process block 808, a sensor-motor map is saved, and the probe is removed. At block 810, extraction of the patient's central nervous system (CNS) (e.g., brain and spinal or Brain only by cutting at level C2 or below) is performed, for example, by following surgical procedures involving laminectomies and brain extraction. Brain extraction will follow procedures that were developed for the cryogenic preservation of the brain. At block 812, a life support machine (e.g., a perfusion pump, oxygenator, waste scrubber, etc.) is connected to the extracted CNS. At block 814, the intraspinal cord probe is implanted and sealing of the extracted CNS is carried out in a sterile environment.

At block 816, the intraspinal probe is connected to the synthetic/robotic body via a through port or wirelessly. At block 818, the synthetic/robotic body is paired to the intraspinal probe via the saved mapping file. This pairing forms the spinal cord machine interface (SCMI). For example, FIGS. 1A-1D show a schematic representation of the SCMI 100, according to various aspects of the present disclosure. In this implementation, a synthetic body 110 is envisioned. The synthetic body 110 is equipped with sensors and actuators 150 (see FIG. 1D) that communicate with a patient's brain 120 (see FIG. 1A) to establish a bi-directional functionality (sensory and motorial). In this implementation, the patient's brain 120 is removed from the patient's biological body and inserted into the synthetic body 110. For example, a life sustaining machine, such as a miniaturized version of an extracorporeal membrane oxygenation, can be employed for this task.

Various aspects of the present disclosure effectively utilize the native functional organization of the lower brainstem/spinal cord to communicate with the patient's brain bi-directionally through custom-made, flat and/or nanopatterned electrodes. The custom-made electrodes exhibit an improved signal-to-noise ratio, charge injection delivery capability, and electrode stability over time. In operation, the white matter in the spinal cord communicates with the brain in one-dimension and is characterized by somatotopy in which representations of motor and sensory activity of the body are discretely and spatially organized in axon bundles. The collection of these axon bundles (occupying ˜1 cm²) holds all the motor and sensory information that is being exchanged between the body and the brain. As a result of this intrinsic map and compactness of information within the spinal cord, stimulation of target areas becomes simpler and a reduced number of electrodes (e.g., fewer than 500 electrodes of ˜30×30 μm² dimensions) are utilized to substantial restore sensation (e.g., over 90%) of the body. In short, the disclosed spinal cord machine interface (SCMI) device is a compelling candidate for developing neural interfaces capable of restoring both sensation and motor control in paralyzed people.

Additionally, the disclosed SCMI provides a platform that is coupled to a variety of sensors and actuators to implement different functions. For example, possible uses of an SCMI platform include pairing the SCMI platform with a bladder volume/pressure sensor and a sacral nerve stimulator to restore micturition in paralyzed patients. Additionally, the SCMI interface may be employed for (1) bowel movement restoration; (3) chronic pain suppression in spinal cord injury patients; (3) restoring sexual functions in paralyzed patients; and (4) restoring mobility in paralyzed patients. For instance, a number of pressure/temperature sensors can be attached to the patient's limbs so that spatially selective information about the location of the stimulus can be relayed to the intraspinal probe which will use pre-mapped channels to selectively stimulate the correct spinal tracts in order to evoke physical sensation in the patient's brain.

Conversely when the patient decides to move a given limb, the signal coming from the patient's brain is detected by the sensing portion of the intraspinal probe and is relayed to either biological or synthetic actuators that lead to movement. The definitive application of the SCMI platform involves providing an alternative to currently terminal diseases such as pancreatic cancer, mesothelioma, end-stage organ failure, and the like. Various aspects of the present disclosure envision transplanting the brain of a terminally diseased person into a robotic body equipped with a life supporting system (e.g., extra-corporeal membrane oxygenation, artificial nutrition, and hydration system). In operation, the SCMI interface is implanted at the base of the patient's brain (e.g., lower pons or upper spinal cord) to enable bi-directional communication between the patient's robotic body and their brain.

For a firmware and/or software implementation, the methodologies may be implemented with modules (e.g., procedures, functions, and so on) that perform the functions described herein. A machine-readable medium tangibly embodying instructions may be used in implementing the methodologies described herein. For example, software codes may be stored in a memory and executed by a processor unit. Memory may be implemented within the processor unit or external to the processor unit. As used herein, the term “memory” refers to types of long term, short term, volatile, nonvolatile, or other memory and is not to be limited to a particular type of memory or number of memories, or type of media upon which memory is stored.

If implemented in firmware and/or software, the functions may be stored as one or more instructions or code on a computer-readable medium. Examples include computer-readable media encoded with a data structure and computer-readable media encoded with a computer program. Computer-readable media includes physical computer storage media. A storage medium may be an available medium that can be accessed by a computer. By way of example, and not limitation, such computer-readable media can include RAM, ROM, EEPROM, CD-ROM or other optical disk storage, magnetic disk storage or other magnetic storage devices, or other medium that can be used to store desired program code in the form of instructions or data structures and that can be accessed by a computer. Disk and disc, as used herein, include compact disc (CD), laser disc, optical disc, digital versatile disc (DVD), and Blu-ray disc, where disks usually reproduce data magnetically, while discs reproduce data optically with lasers. Combinations of the above should also be included within the scope of computer-readable media.

In addition to storage on computer-readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims.

Although the present disclosure and its advantages have been described in detail, it should be understood that various changes, substitutions, and alterations can be made herein without departing from the technology of the disclosure as defined by the appended claims. For example, relational terms, such as “above” and “below” are used with respect to a substrate or electronic device. Of course, if the substrate or electronic device is inverted, above becomes below, and vice versa. Additionally, if oriented sideways, above, and below may refer to sides of a substrate or electronic device. Moreover, the scope of the present application is not intended to be limited to the particular configurations of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding configurations described herein may be utilized, according to the present disclosure. Accordingly, the appended claims are intended to include within their scope such processes, machines, manufacture, compositions of matter, means, methods, or steps.

Those of skill would further appreciate that the various illustrative logical blocks, modules, circuits, and algorithm steps described in connection with the disclosure herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The various illustrative logical blocks, modules, and circuits described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, multiple microprocessors, one or more microprocessors in conjunction with a DSP core, or any other such configuration.

The steps of a method or algorithm described in connection with the disclosure may be embodied directly in hardware, in a software module executed by a processor, or in a combination of the two. A software module may reside in RAM, flash memory, ROM, EPROM, EEPROM, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In one or more exemplary designs, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a general purpose or special purpose computer. By way of example, and not limitation, such computer-readable media can include RAM, ROM, EEPROM, CD-ROM or other optical disk storage, magnetic disk storage or other magnetic storage devices, or any other medium that can be used to carry or store specified program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD) and Blu-ray disc where disks usually reproduce data magnetically, while discs reproduce data optically with lasers. Combinations of the above should also be included within the scope of computer-readable media.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. A phrase referring to “at least one of” a list of items refers to any combination of those items, including single members. As an example, “at least one of: a, b, or c” is intended to cover: a; b; c; a and b; a and c; b and c; and a, b, and c. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “a step for.”

What is claimed is:

1. A spinal cord machine interface (SCMI) device, comprising:
an intraspinal probe, comprising at least one implantable shank including a sensing electrode array and a stimulating electrode array; and
an application specific integrated circuit (ASIC) configured to detect action potentials from an efferent portion of a patient's spinal cord using the sensing electrode array and to stimulate an afferent portion of the patient's spinal cord using the stimulating electrode array according to a functional map linking specific regions of the spinal cord to their respective sensory and motor functions.

2. The SCMI device of claim 1, in which the sensing electrode array comprises a plurality of two-dimensional (2D) flat electrodes arranged in a 2D matrix in a first direction and a second direction.

3. The SCMI device of claim 2, in which the plurality of 2D flat electrodes are coated with a conductive material, comprising titanium nitride (TiN), gold (Au), and/or platinum (Pt).

4. The SCMI device of claim 1, in which the stimulating electrode array comprises a plurality of three-dimensional (3D) electrodes arranged in a 2D matrix in a first direction and a second direction.

5. The SCMI device of claim 4, in which the plurality of 3D electrodes comprise a conductive region surrounding an electrode post.

6. The SCMI device of claim 4, in which the plurality of 3D electrodes are coated in a conductive material, comprising titanium (Ti)/Au (Ti/Au), Ti/Pt (Ti/Pt), Ti/Pt iridium (Ir) (Ti/PtIr), Ti/Pt/Ir, Ti/Pt/Ir oxide (Ox) (Ti/Pt/TrOx), Ti/Ir, Ti/Ti nitride (N) (Ti/TiN), Ti/Pt/TiN, and/or Ti/IrOx.

7. The SCMI device of claim 4, in which the plurality of 3D electrodes comprise a plurality of nanopatterned electrodes.

8. The SCMI device of claim 7, in which the plurality of nanopatterned electrodes comprises black silicon (BSi) having a predetermined shape.

9. The SCMI device of claim 8, in which the predetermined shape comprises silicon grass or needle-like morphology.

10. The SCMI device of claim 1, in which the ASIC comprises:
an intraspinal sensing module;
an intraspinal stimulation module;
a control module coupled to the intraspinal sensing module and the intraspinal stimulation module;
a receiving module; and
a transmitting module, in which the action potentials detected by the intraspinal sensing module are transmitted to an external actuator to carry out a brain-initiated task.

11. A method for a central nervous system (CNS) transfer, the method comprising:
implanting a bi-directional intraspinal cord probe in a target area at a cervical level of a patient;
performing sensory and motor mapping to establish a map between an electrode site of the bi-directional intraspinal cord probe and a corresponding physiological function of the patient;
transplanting the central nervous system (CNS) of the patient to a synthetic body; and
forming a spinal cord machine interface (SCMI) to the CNS in the synthetic body.

12. The method of claim 11, in which implanting comprises:
performing spinal surgery on the patient at the cervical vertebral level;
identifying the target area at the cervical vertebral level of the patient to implant the bi-directional intraspinal cord probe; and
marking the identified target area.

13. The method of claim 11, in which the performing of the sensory and motor mapping comprises:
recording, by the electrode site of the bi-directional intraspinal cord probe, a movement of a specified limb from spinal motor tracts of the patient; and
repeating the recording for each muscle of the patient to form the sensory and motor mapping of the map, in which the map enables interpreting of a patient's motor intentions and selectively stimulate sensory responses corresponding to different areas of the body.

14. The method of claim 11, in which the performing sensory and motor mapping comprises:
activating each electrode of the bi-directional intraspinal cord probe in a Fasciculus Cuneatus and a Fasciculus Gracilis of the patient;
recording responses of the patient during the activating as a sensory map; and
rasterizing the sensory map across a body of the patient.

15. The method of claim 11, in which two-point discrimination is performed to determine points of the body of the patient in which the mapping is performed, in which the map enables a target action according to a location of action potentials in a patient's spinal cord and a learned mapping of a location of the action potentials to the target action.

16. The method of claim 11, in which transplanting comprises:
extracting the CNS of a patent thus preventing brain death of the patient; and
connecting a life support machine to the extracted CNS.

17. The method of claim 16, further comprising:
implanting the bi-directional intraspinal probe into the extracted CNS; and
sealing the CNS and the bi-directional intraspinal probe in a sterile environment.

18. The method of claim 17, in which the implanting is performed according to markers established during an initial implant of the bi-directional intraspinal cord probe.

19. The method of claim 11, in which transplanting comprises:
connecting the bi-directional intraspinal probe to the synthetic body; and
pairing the synthetic body to the intraspinal probe using the map between the electrode site of the bi-directional intraspinal cord probe and the corresponding physiological function of the patient.

20. A method for bladder control, the method comprising:
monitoring, using a bladder sensing unit, a bladder status until the bladder status indicates a bladder volume within a predetermined percentage of a predetermined maximum bladder volume;
stimulating, using a stimulator electrode array of an intraspinal probe, a patient for a predetermined amount of time to indicate the bladder status regarding the bladder volume within the predetermined percentage of the predetermined maximum bladder volume;
detecting, using a sensing electrode array of the intraspinal probe, action potentials in an efferent/motor micturition center of the patient; and
activating a sacral nerve anterior root stimulator (SARS) to void a bladder of the patient in response to the detecting.
